(19) United States
(12) Patent Application Publication
(10) Pub. No.: US 2023/0416191 A1
(43) Pub. Date: Dec. 28, 2023

(54) Compounds, Compositions and Methods for the Treatment of CREB-dependent Diseases

(71) Applicant: Sereno Sciences, Inc., San Carlos, CA (US)

(72) Inventors: Leo Gu, Los Gatos, CA (US); Narinder S. Banait, San Carlos, CA (US)

(21) Appl. No.: 17/846,492

(22) Filed: Jun. 22, 2022

(51) Int. Cl. C07C 237/48

(52) U.S. Cl. CPC C07C 237/48

(57) ABSTRACT

The present invention provides bicyclic compounds, pharmaceutical formulations of the compounds, and methods of using the formulations in the treatment or prevention of cancers mediated by cyclic-AMP (cAMP) response element binding protein (CREB), such as leukemias, breast cancer, lung cancer, and prostate cancer.

### FIELD OF INVENTION

The present invention relates to novel compounds that modulate cyclic-AMP response element binding (CREB) protein, pharmaceutical compositions comprising these compounds, and methods for the treatment of CREB-dependent diseases.

### BACKGROUND

Cyclic-AMP (cAMP) response element binding protein (CREB) is a ˜44 kDa basic leucine zipper (bZIP)-containing transcription factor that is localized in the nucleus. cDNA clones for human CREB were isolated in 1988 (Hoeffler J P, et al. (1988) Cyclic AMP-responsive DNA-binding protein: structure based on a cloned placental cDNA. Science. 242 (4884): 1430-3). The deduced 326-amino acid protein is composed of specific structural components characterized by a transactivation domain that consists of a kinase inducible domain (KID), a constitutively active glutamine-rich domain (Q2) that synergize in response to cAMP stimulation, and the leucine zipper that may bind DNA or DNA-associated proteins.

CREB activates transcription of target genes in response to a diverse array of stimuli including peptide hormones, growth factors, PKA, mitogen-activated protein kinases (MAPKs) and calcium calmodulin-dependent protein kinases (CaMKs). These kinases all phosphorylate CREB at serine 133, an event required for CREB-mediated transcription. Ser-133 phosphorylation promotes target gene activation in part by means of recruitment of the coactivator paralogs CREB-binding protein (CBP)/p300. Activated CREB binds genomic DNA at loci possessing the consensus the cAMP Response Element or ‘CRE’ site. Initiation of CREB-driven transcription at these loci requires that CREB recruits and binds a co-activator, the histone acetyltransferase CREB Binding Protein (CBP) through the interaction of phosphorylated KID with the KID Interacting (KIX) domain. The CREB/CBP complex triggers local histone acetylation and subsequent recruitment of the RNA polymerase transcriptional machinery to the promoter resulting in the expression of CREB-driven genes that regulate cell proliferation and survival.

CREB is overexpressed in several cancers. CREB is overexpressed in about 60% of acute myeloid leukemia (AML) patients and is associated with a significantly worse prognosis and an increased risk of relapse compared to patients with basal CREB expression. CREB overexpression in AML cells augments their growth rate and confers resistance to apoptosis. Conversely, CREB knockdown in AML cells decreases cell proliferation and induces apoptosis without any effects on long-term engraftment of hematopoietic stem cells (HSCs). Overexpression of CREB was also seen in cancer tissues from breast cancer patients, non-small-cell lung cancer (NSCLC) patients, pancreatic cancer and Hepatocellular carcinoma (HCC).

U.S. Pat. No. 5,919,649 discloses DNA sequences encoding CREB and methods directed towards enhancing expression of CREB by transforming a host cell with a construct containing a CREB gene.

U.S. Pat. No. 8,653,086 discloses naphthols, naphthol AS-E in particular, as inhibitors of CREB. Several later publications have noted that one of the major issues with napththol AS-E is its low bioavailability.

U.S. Pat. No. 9,073,820 discloses naphthamide and quinoline carboxamide compounds as inhibitors of CREB. The naphthamide compound had a half-life of 9.6 hours and oral bioavailability of only 7.1%.

PCT Publication WO2017156489, also published as U.S. Publication No. 2021/05292, discloses naphthamide and salicylamide based compounds as inhibitors of CREB. The publication discloses that N-(4-Cyanophenyl)-3-Hydroxy-2-naphthamide, designated as Compound A, was useful for the treatment of leukemias.

### SUMMARY

The present invention provides novel compounds, pharmaceutical formulations comprising the compounds, and method for treating or preventing a cancer by administering the formulations.

The disclosed compounds comprising the formula:

[symbol]

wherein R₁, R₂, R₃, R₄, and R₅ are independently selected to be H, Cl, F, Br, I, CN, or NO₂, X is —NH—C(O)—, —C(O)—, or —O—, n is 1, 2, 3, or 4, m is 3 or 4, and its salts, solvates, and prodrugs thereof.

In another aspect, provided are compounds comprising the formula:

[symbol]

wherein R₆, R₇, and R₈ are independently selected to by H, Cl, F, Br, I, CN, or NO₂, and its salts, solvates, and prodrugs thereof.

In another aspect, provided are compounds comprising the formula:

[symbol]

wherein R₉, R₁₀, R₁₁, R₁₂, and R₁₃ are independently selected to be H, Cl, F, Br, I, CN, or NO₂, n is 1, 2, 3, or 4, m is 3 or 4, and its salts, solvates, and prodrugs thereof.

In another aspect, provided are pharmaceutical compositions comprising a compound of formula:

[symbol]

wherein R₁, R₂, R₃, R₄, and R₅ are independently selected to be H, Cl, F, Br, I, CN, or NO₂, X is —NH—C(O)—, —C(O)—, or —O—, n is 1, 2, 3, or 4, m is 3 or 4, and its salts, solvates, and prodrugs thereof, and a pharmaceutically acceptable carrier.

In another aspect, provided are method of treating or preventing a cancer in a patient in need thereof, the method comprising administering to a patient a therapeutically effective amount of a compound that inhibits Cyclic-AMP (cAMP) response element binding protein (CREB).

These and other aspects of the present invention will become evident upon reference to the following detailed description.

### DETAILED DESCRIPTION

### I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (2004) “Advanced Organic Chemistry 4^{rd} Ed.” Vols. A and B, Springer, New York. The practice of the present invention will employ, unless otherwise indicated, conventional methods of mass spectroscopy, protein chemistry, biochemistry, and pharmacology, within the skill of the art.

The term “agonist” means a molecule such as a compound, a drug, an enzyme activator or a hormone that enhances the activity of another molecule or the activity of the target receptor.

The term “antagonist” means a molecule such as a compound, a drug, an enzyme inhibitor, or a hormone, that diminishes or prevents the action of another molecule or the activity of the target receptor.

The terms “effective amount” or “pharmaceutically effective amount” refer to a sufficient amount of the agent to provide the desired biological result without an unacceptable toxic effect. That result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined by one of ordinary skill in the art using routine experimentation.

As used herein, the terms “treat” or “treatment” are used interchangeably and are meant to ameliorating the disease or disorder (i.e., arresting or reducing the development of the disease or at least one of the clinical symptoms thereof). In one embodiment “treating” or “treatment” refers to ameliorating at least one symptoms of the disease. In another embodiment, “treating” or “treatment” refers to inhibiting the disease or disorder, either physically (e.g., stabilization of a discernible symptom), physiologically, (e.g., stabilization of a physical parameter), or both.

As used herein, the term “mammal subject” encompasses any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “modulator” means a molecule that interacts with a target. The interactions include, but are not limited to, agonist, antagonist, and the like, as defined herein.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

The term “pharmaceutically acceptable salt” of a compound means a salt that is pharmaceutically acceptable and that possesses the desired pharmacological activity of the parent compound. Such salts, for example, include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1 -carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like;
(2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

As used herein, the term “patient” encompasses any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

All publications, patents and patent applications cited herein, whether supra or infra, are hereby incorporated by reference in their entirety.

### II. Description of the Invention

The compositions and methods of the present invention modulate cyclic-AMP response element binding (CREB) pathway signaling. Thus, the present invention provides compounds, compositions and methods for the prevention or treatment of a CREB mediated condition or disease. The CREB pathway is a critically important pathway, the expression of which is increased in patients with diseases, such as leukemias, breast cancer, lung cancer, prostate cancer, hepatocellular carcinoma (HCC), and the like. Therefore, modulating CREB expression in patients with these diseases can prevent or reverse the disease.

### Compounds

The disclosed compounds comprising the formula:

[symbol]

wherein R₁, R₂, R₃, R₄, and R₅ are independently selected to be H, Cl, F, Br, I, CN, or NO₂, X is —NH—C(O)—, —C(O)—, or —O—, n is 1, 2, 3, or 4, m is 3 or 4. Exemplary compounds of are given in the table below.

| Compound | R1 | R2 | R3 | R4 | R5 | n | m | X |
|---|---|---|---|---|---|---|---|---|
| SRS1001 | H | H | H | H | H | 2 | 3 | HNC(O) |
| SRS1002 | H | H | Cl | H | H | 2 | 3 | HNC(O) |
| SRS1003 | H | H | F | H | H | 2 | 3 | HNC(O) |
| SRS1004 | H | H | CN | H | H | 2 | 3 | HNC(O) |
| SRS1005 | Cl | H | Cl | H | H | 2 | 3 | HNC(O) |
| SRS1006 | F | H | H | H | H | 2 | 3 | HNC(O) |
| SRS1007 | F | H | H | H | F | 2 | 3 | HNC(O) |
| SRS1008 | F | H | F | H | F | 2 | 3 | HNC(O) |
| SRS1009 | F | H | F | H | H | 2 | 4 | HNC(O) |
| SRS1010 | F | H | F | H | H | 2 | 3 | O |
| SRS1011 | Cl | H | Cl | H | H | 2 | 3 | O |
| SRS1012 | H | F | H | F | H | 2 | 3 | O |
| SRS1013 | H | H | H | H | H | 2 | 3 | O |
| SRS104 | H | H | Cl | H | H | 2 | 3 | O |
| SRS1015 | H | H | F | H | H | 2 | 3 | O |
| SRS1016 | H | H | CN | H | H | 2 | 3 | O |

In the compounds above, R1 and R3 cannot be F when R2, R₄, and R5 are H, X is HNC(O), n is 2 and m is 3.

In another aspect, provided are compounds comprising the formula:

[symbol]

wherein R₆, R₇, and R₈ are independently selected to by H, Cl, F, Br, I, CN, or NO₂.

In another aspect, provided are compounds comprising the formula:

[symbol]

wherein R₉, R₁₀, R₁₁, R₁₂, and R₁₃ are independently selected to be H, Cl, F, Br, I, CN, or NO₂, n is 1, 2, 3, or 4,m is 3 or 4.

The present invention also provides prodrugs of the compounds described in detail above wherein the prodrug converts in vivo to the compound. A prodrug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound that contain one or more protective groups and are converted to an active form by metabolism or solvolysis. Both the active drug form and any released metabolic products should have acceptably low toxicity.

Exemplary prodrugs are, for example, esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Any compound given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds as defined above include isotopes of hydrogen, carbon, nitrogen, oxygen, phosphorous, fluorine, and chlorine, such as ²H, ³H, ¹¹C, ¹³C, ¹⁴C , ¹⁵N , ⁸F, ³¹P , ³²p, ³⁵S, ³⁶Cl, ¹²⁵I respectively. Isotopically labeled compounds of this invention and prodrugs thereof can generally be prepared by carrying out the synthetic procedures by substituting a readily available isotopically labeled reagent for a non-isotopically labeled reagent. Isotopically-labeled compounds can generally be prepared by conventional techniques known to those skilled in the art using an appropriate isotopically-labeled reagents in place of the non-labeled reagent previously employed.

The compounds disclosed above, in free form, may be converted into salt form, and vice versa, in a conventional manner understood by those skilled in the art. The compounds in free or salt form can be obtained in the form of hydrates or solvates containing a solvent used for crystallization. The compounds can be recovered from reaction mixtures and purified in a conventional manner. Pharmaceutically acceptable solvates in accordance with the invention include those wherein the solvent of crystallization may be isotopically substituted, e.g. D₂O, d₆-acetone, d₆-DMSO.

The compounds of the invention described in detail above were designed to have increased bioavailability. Thus, the compounds have oral bioavailability that is >20%, preferably >30%, more preferably >40%. Without being bound to theory, the reason for the very low oral bioavailability of the naphthamide and quinoline carboxamide CREB inhibitors described in U.S. Pat. No. 9,073,820 is that the phenolic hydroxyl group has a low pKa such that it ionizes under the basic conditions found in the small intestines, which produces an insoluble zwitterion thereby leading to low bioavailability. Thus, the compounds of the invention do not have an ionizable group, such as a phenolic hydroxyl group, as a substitutent.

### IV. Formulations

The compounds described above are preferably used to prepare a medicament, such as by formulation into pharmaceutical compositions for administration to a subject using techniques generally known in the art. A summary of such pharmaceutical compositions may be found, for example, in Remington's Pharmaceutical Sciences, Mack Publishing Co., Easton, PA. The compounds of the invention can be used singly or as components of mixtures. Preferred forms of the compounds are those for systemic administration as well as those for topical or transdermal administration. Formulations designed for timed release are also with the scope of the invention. Formulation in unit dosage form is also preferred for the practice of the invention.

In unit dosage form, the formulation is divided into unit doses containing appropriate quantities of one or more compound. The unit dosage may be in the form of a package containing discrete quantities of the formulation. Non-limiting examples are packeted tablets or capsules, and powders in vials or ampoules.

The compounds of the invention may be labeled isotopically (e.g. with a radioisotope) or by another other means, including, but not limited to, the use of chromophores or fluorescent moieties, bioluminescent labels, or chemiluminescent labels. The compositions may be in conventional forms, either as liquid solutions or suspensions, solid forms suitable for solution or suspension in a liquid prior to use, or as emulsions. Suitable excipients or carriers are, for example, water, saline, dextrose, glycerol, alcohols, aloe vera gel, allantoin, glycerin, vitamin A and E oils, mineral oil, propylene glycol, PPG-2 myristyl propionate, and the like. Of course, these compositions may also contain minor amounts of nontoxic, auxiliary substances, such as wetting or emulsifying agents, pH buffering agents, and so forth.

Methods for the preparation of compositions comprising the compounds of the invention include formulating the derivatives with one or more inert, pharmaceutically acceptable carriers to form either a solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein.

A carrier of the invention can be one or more substances which also serve to act as a diluent, flavoring agent, solubilizer, lubricant, suspending agent, binder, or tablet disintegrating agent. A carrier can also be an encapsulating material.

In powder forms of the invention's compositions, the carrier is preferably a finely divided solid in powder form which is interdispersed as a mixture with a finely divided powder from of one or more compound. In tablet forms of the compositions, one or more compounds is intermixed with a carrier with appropriate binding properties in suitable proportions followed by compaction into the shape and size desired. Powder and tablet form compositions preferably contain between about 5 to about 70% by weight of one or more compound. Carriers that may be used in the practice of the invention include, but are not limited to, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The compounds of the invention may also be encapsulated or microencapsulated by an encapsulating material, which may thus serve as a carrier, to provide a capsule in which the derivatives, with or without other carriers, is surrounded by the encapsulating material. In an analogous manner, cachets comprising one or more compounds are also provided by the instant invention. Tablet, powder, capsule, and cachet forms of the invention can be formulated as single or unit dosage forms suitable for administration, optionally conducted orally.

If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Formulations for parenteral administration may be in the form of aqueous or non-aqueous sterile injection solutions or suspensions. These solutions and suspensions can be prepared from sterile powders or granules having one or more of the carriers or diluents mentioned for use in the formulations for oral administration. The compounds can be dissolved or suitably emulsified in water, polyethylene glycol, propylene glycol, ethanol, corn oil, cottonseed oil, peanut oil, sesame oil, benzyl alcohol, sodium chloride, and/or various buffers. Other adjuvants and modes of administration are widely known in the pharmaceutical art.

For oral administration, the pharmaceutical composition can be in the form of, for example, a tablet, capsule, a soft gelatin (softgel) capsule, a hard gelatin capsule, suspension or liquid. The pharmaceutical composition is preferably made in the form of a dosage unit containing a particular amount of the active ingredient. Examples of such dosage units are tablets or softgel capsules. The active ingredient can also be administered by injection as a composition wherein, for example, saline, dextrose or water can be used as a suitable carrier.

Soft gelatin capsules can be prepared in which capsules contain a mixture of a CREB inhibitor and at least one other active compound, and oleaginous and/or non-aqueous, and/or water miscible solvents such as polyethylene glycol and the like. Hydrophilic solvents compatible with softgel capsules can include PEG400, PEG800, ethanol, glycerin, PPG, polysorbates, povidone (PVP), and the like containing up to about 5-8% water. The softgel capsules can optionally contain a buffer, a co-solvent, or a nucleophile. Hard gelatin capsules can contain mixtures of CREB inhibitor and at least one other active compound in combination with a solid, pulverulent carrier, such as, for example, lactose, saccharose, sorbitol, mannitol, potato starch, corn starch, amylopectin, cellulose derivatives, or gelatin.

In suppository forms of the compositions, a low-melting wax such as, but not limited to, a mixture of fatty acid glycerides, optionally in combination with cocoa butter is first melted. One or more compounds are then dispersed into the melted material by, as a non-limiting example, stirring. The non-solid mixture is then placed into molds as desired and allowed to cool and solidify.

Non-limiting compositions in liquid form include solutions suitable for oral or parenteral administration, as well as suspensions and emulsions suitable for oral administration. Sterile aqueous based solutions of one or more compounds, optionally in the presence of an agent to increase solubility of the derivative(s), are also provided. Non-limiting examples of sterile solutions include those comprising water, ethanol, and/or propylene glycol in forms suitable for parenteral administration. A sterile solution of the invention may be prepared by dissolving one or more compounds in a desired solvent followed by sterilization, such as by filtration through a sterilizing membrane filter as a non-limiting example. In another embodiment, one or more compounds are dissolved into a previously sterilized solvent under sterile conditions.

A water-based solution suitable for oral administration can be prepared by dissolving one or more compounds in water and adding suitable flavoring agents, coloring agents, stabilizers, and thickening agents as desired. Water based suspensions for oral use can be made by dispersing one or more compounds in water together with a viscous material such as, but not limited to, natural or synthetic gums, resins, methyl cellulose, sodium carboxymethyl cellulose, and other suspending agents known to the pharmaceutical field.

Pulmonary administration can be achieved by inhalation or by the introduction of a delivery device into the pulmonary system, e.g., by introducing a delivery device which can dispense (wet or dry) the pharmaceutical composition. The CREB inhibitor or its combination with at least one other active compound can be provided in a dispenser which delivers the composition in a form sufficiently small such that it can be inhaled. The CREB inhibitor or its combination can be provided in measured doses, in a dispenser that delivers a metered dose, or a dry powder inhaler.

In therapeutic use, the compounds of the invention are each administered to a subject at a dosage level of from about 0.05-8, 0.05-80, 0.5-8, or 0.5-80 mg/kg, of body weight per day. For example, in a human subject of approximately 70 kg, this is a dosage of from 4 mg to 600 mg per day. Such dosages, however, may be altered depending on a number of variables, not limited to the activity of the compound used, the condition to be treated, the mode of administration, the requirements of the individual subject, the severity of the condition being treated, and the judgment of the practitioner.

The foregoing ranges are merely suggestive, as the number of variables in regard to an individual treatment regime is large, and considerable excursions from these recommended values are not uncommon.

### V. Methods of Use

A compound of the invention can be administered to a subject upon determination of the subject as having a disease caused by the overexpression of CREB, such as leukemia, triple negative breast cancer, HCC, lung cancer, or prostate cancer, or unwanted condition that would benefit by treatment with said derivative. The determination can be made by medical or clinical personnel as part of a diagnosis of a disease or condition in a subject.

For administration to non-human animals, the drug or a pharmaceutical composition

containing the drug may also be added to the animal feed or drinking water. It will be convenient to formulate animal feed and drinking water products with a predetermined dose of the drug so that the animal takes in an appropriate quantity of the drug along with its diet. It will also be convenient to add a premix containing the drug to the feed or drinking water approximately immediately prior to consumption by the animal.

### Kits/Articles of Manufacture

For use in the therapeutic applications described herein, kits and articles of manufacture are also within the scope of the invention. Such kits can comprise a carrier, package, or container that is compartmentalized to receive one or more containers such as vials, tubes, and the like, each of the container(s) comprising one of the separate elements to be used in a method of the invention. Suitable containers include, for example, bottles, vials, syringes, and test tubes. The containers can be formed from a variety of materials such as glass or plastic.

For example, the container(s) can comprise one or more compounds of the invention, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods of the present invention.

A kit of the invention will typically comprise one or more additional containers, each with one or more of various materials (such as reagents, optionally in concentrated form, and/or devices) desirable from a commercial and user standpoint for use of a compound of the invention. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

The terms “kit” and “article of manufacture” may be used as synonyms.

Having now generally described the invention, the same will be more readily understood through reference to the following examples which are provided by way of illustration and are not intended to be limiting of the present invention, unless specified.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The compounds were synthesized using methods described in Xie et al. J. Med. Chem. 2015, 58, 5075-5087, using the general synthetic scheme shown below: The commercially available 3-hydroxy-2-naphthoic acid 1 was converted into methyl ester 2, which was then subjected to Mitsunobu reaction with Boc- protected amino-alcohol to generate the ester 3. In general, to a solution of phenol 2 (1 molar equivalent), Boc protected ethanol amine (1.2-1.5 molar equivalent), and PPh₃ (1.2-1.5 molar equivalent) in TRF was added diethyl azodicaroxylate (DEAD) (1.2-1.5 molar equivalent) in TRF dropwise at 0° C. The reaction mixture was stirred at room temperature overnight. The solvent was removed under reduced pressure and the residue was purified by silica gel flash column chromatography to give the corresponding product 3.

Saponication of 3 gave acid 4. Generally, to a solution of methyl ester (1 molar equivalent) in MeOH:TEEF:H₂O (1:1:1) was added LiOH·H₂O (5 molar equivalent) at room temperature. The resulting mixture was stirred at room temperature overnight. The organic solvents were removed under reduced pressure, and the residue was acidified with 2 N HCl at 0° C. to pH 2. The reaction mixture was extracted with ethyl acetate. The organic layer was separated and washed with brine and dried over Na₂SO₄. The solution was filtered, and the solvent was evaporated to give the acid 4.

The acid 4 was then coupled with the appropriated substituted aniline 5 to yield amide 6. Generally, to a stirred solution of 4 (1 molar equivalent) and TEA (1.0 molar equivalent) in THF was added MSCl (1 molar equivalent) dropwise at 0° C. The reactionl mixture was stirred at 0° C. for 30 min, and then substituted aniline 5 (1 molar equivalent) was added. The reaction mixture was stirred at room temperature overnight. The reaction mixture was diluted with 5% NaHCO₃ and extracted with ethyl acetate. The organic layer was separated, washed with brine, and dried over Na₂SO₄. The solution was filtered, and the solvent was removed to give a residue, which was purified by silica gel flash column chromatography to yield the corresponding amide 6.

The Boc group of 6 was then deprotected under acidic condition to generated the free amine, and amide coupling with 7 followed by removal of the Boc group under acidic condition provided the final compound 8. The NMR was obtained in CD₃OD using a Varian 400 mHz.

### General Procedure for Inhibition of CREB-Mediated Gene Transcription

The compounds were tested for their inhibition of CREB-mediated gene transcription according to the method described in Xie et al. J. Med. Chem. 2015, 58, 5075-5087. Generally, HEK293T cell transfected with luciferase reporter cell lines under CRE control were obtained from commercial sources. The cells were plated into 96-well plates (1-2×10 cells/well). The cells were allowed to attach to the bottom of the wells overnight, when compounds of different concentrations were added to the cells. Forskolin (10 uM, LC Laboratories, Woburn, Mass.) was added 30 min after the addition of the compounds. The cells were then incubated at 37° C. for 5 h. The media in the wells were removed and the cells were then lysed in 30 u1 of 1×Renilla luciferase lysis buffer (Promega, Madison, Wis.). To measure Renilla luciferase activity, five uL of the lysate was combined with 30 uI of benzyl-coelenterazine (Nanolight, Pinetop, Ariz.) solution in PBS (pH 7.4, 10 ug/mL). The sample protein concentration was determined by Dye Reagent Concentrate (Bio-Rad, Hercules, Calif). The Renilla luciferase activity was normalized to protein content in each well and expressed as relative luciferase unit/ug protein (RLU/ug protein). The IC₅₀ was derived from non-linear regression analysis of the RLU/ug protein-concentration curve.

### General Procedure for the Cytotoxicity Assay

The cells were obtained from ATCC, IMDM was obtained from Lonza, and FBS was obtained from Hyclone. Cell-titer-Glow for cytotoxicity was from Promega. Sterile 96-well tissue culture plates were from Corning. Different concentrations of the test compounds were tested in triplicate wells of either 96-well or 384-well tissue culture plates. The screening was repeated two additional times.

The cells from ATCC were thawed, washed once with complete media, and cultured in T75 flask in a 37C CO2 incubator. Cell density was maintained between 5×10 5 to 1×10⁶ cells/ml.

Compounds were individually weighed and dissolved in DMSO to obtain 10 mM solutions of each and stored at −20° C. until used. To select concentrations of the compounds for cytotoxicity, all the compounds were initially tested at 100 μM and 30 μM in a 48 hr cytotoxicity assay.

Two microliter of each concentration of the test compounds were transferred into triplicate wells of a 96-well tissue culture plate. Into each well 100 μof the cell suspension was added, the plate was lidded and incubated at 37° C. in a 5% CO₂ incubator maintained at 95% humidity

After 72hr of incubation, the plate was removed from the incubator, and the cell viability was determined using Cell-titer-glow reagent as per the protocol described in the kit manual. Signals from the plate was measured using Victor-II plate reader. The raw data from the plate reader were exported to Excel. For each concentration of the drug tested the average signal and SD from the triplicate wells, as well as the percent cytotoxicity were calculated. The resulting data were exported to Prism-graph-pad, the % cytotoxicity was plotted against log concentration and the resulting curves were fitted using non-linear dose response fit and the resulting IC50 values were extracted.

### Example 1

### Synthesis of SRS-1001

The structure of the SRS-1001 is given below:

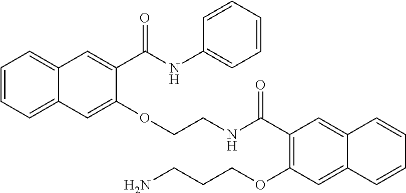 [SMILES: NCCCOc1cc2ccccc2cc1C(=O)NCCOc1cc2ccccc2cc1C(=O)Nc1ccccc1]

The compound was synthesized as illustrated in the general scheme above, and isolated as a yellow solid. ¹H NMR (400 MHz, CD₃OD) δ8.27 (s, 1H), 8.01 (s, 1H), 7.88 (d, 3H), 7.84 (d, 1H), 7.61 (d, 2H), 7.51 (m, 3H), 7.41 (m, 3H), 7.32 (t, 2H), 7.03 (t, 1H), 4.54 (t, 2H), 4.23 (t, 2H), 3.94 (t, 2H), 3.29 (t, 2H), and 3.10 (t, 2H). Mass spectrum identified a molecular ion at 534.45 (M+H)⁺. The purity of the compound measured by NMR was found to be >95% and by HPLC was found to be >99%. The IC50 for CREB inhibition was 1.26 μM.

### Example 2

### Synthesis of SRS-1002

The structure of the SRS-1002 is given below:

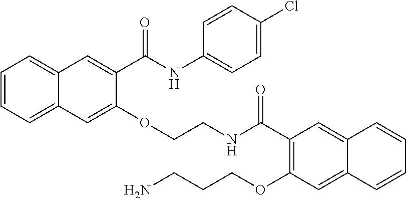 [SMILES: NCCCOc1cc2ccccc2cc1C(=O)NCCOc1cc2ccccc2cc1C(=O)Nc1ccc(Cl)cc1]

The compound was synthesized as illustrated in the general scheme above, and isolated as an off-white solid. ¹H NMR (400 MHz, CD₃OD) δ 8.24 (s, 1H), 7.99 (s, 1H), 7.70 (m, 3H), 7.55 (d, 1H), 7.49 (d, 5H), 7.41 (m, 2H), 7.35 (s, 1H), 7.08 (t, 2H), 4.53 (t, 2H), 4.23 (t, 2H), 3.91 (t, 2H), 3.12 (t, 2H), and 3.12 (t, 2H). Mass spectrum identified a molecular ion at 568.40 (M+H)⁺. The purity of the compound measured by NMR was found to be >95% and by HPLC was found to be >97%. The IC50 for CREB inhibition was 1.55 μM.

### Example 3

Synthesis of SRS-1003

The structure of the SRS-1003 is given below:

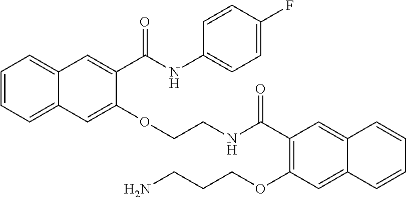 [SMILES: NCCCOc1cc2ccccc2cc1C(=O)NCCOc1cc2ccccc2cc1C(=O)Nc1ccc(F)cc1]

The compound was synthesized as illustrated in the general scheme above, and isolated as an off-white solid. ¹H NMR (400 MHz, CD₃OD) δ8.26 (s, 1H), 8.00 (s, 1H), 7.83 (m, 4H), 7.56 (m, 5H), 7.37 (m, 3H), 6.88 (t, 2H), 4.54 (t, 2H), 4.26 (t, 2H), 3.93 (t, 2H), 3.13 (t, 2H), and 2.12 (t, 2H). Mass spectrum identified a molecular ion at 552.40 (M+H)⁺. The purity of the compound measured by NMR was found to be >99% and by HPLC was found to be >99%. The IC50 for CREB inhibition was 0.41 μM.

### Example 4

### Synthesis of SRS-1004

The structure of the SRS-1004 is given below:

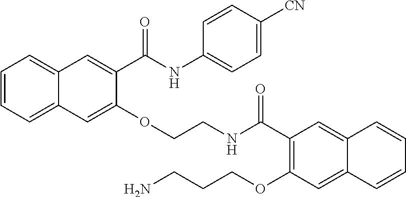 [SMILES: N#Cc1ccc(NC(=O)c2cc3ccccc3cc2OCCNC(=O)c2cc3ccccc3cc2OCCCN)cc1]

The compound was synthesized as illustrated in the general scheme above, and isolated as an off-white solid. ¹H NMR (400 MHz, CD₃OD) δ8.26 (s, 1H), 7.97 (s, 1H), 7.85 (m, 6H), 7.53 (m, 3H), 7.41 (m, 4H), 7.33 (t, 1H), 4.55 (t, 2H), 4.27 (t, 2H), 3.97 (t, 2H), 3.15 (t, 2H), and 2.17 (t, 2H). Mass spectrum identified a molecular ion at 559.45 (M+H)⁺. The purity of the compound measured by NMR was found to be >95% and by HPLC was found to be >99%. The IC50 for CREB inhibition was 1.20 μM.

### Example 5

### Synthesis of SRS-1005

The structure of the SRS-1005 is given below:

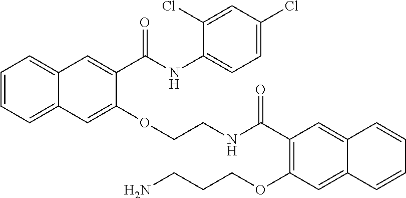 [SMILES: NCCCOc1cc2ccccc2cc1C(=O)NCCOc1cc2ccccc2cc1C(=O)Nc1ccc(Cl)cc1Cl]

The compound was synthesized as illustrated in the general scheme above, and isolated as an off-white solid. ¹NMR (400 MHz, CD₃OD) δ8.64 (s, 1H), 8.23 (d, 1H), 7.91 (m, 3H), 7.70 (d, 1H), 7.59 (s, 1H), 7.50 (m, 4H), 7.3 (m, 3H), 7.17 (d, 1H), 4.80 (t, 2H), 4.72 (t, 2H), 4.27 (t, 2H), 3.30 (t, 2H), and 2.18 (t, 2H). Mass spectrum identified a molecular ion at 602.35 (M+H)⁺. The purity of the compound measured by NMR was found to be >95% and by HPLC was found to be >99%. The IC50 for CREB inhibition was 1.55 μM.

### Example 6

### Synthesis of SRS-1006

The structure of the SRS-1006 is given below:

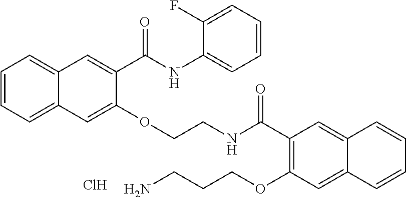 [SMILES: NCCCOc1cc2ccccc2cc1C(=O)NCCOc1cc2ccccc2cc1C(=O)Nc1ccccc1F ; Cl]

The compound was synthesized as illustrated in the general scheme above, and isolated as a white solid. ¹H NMR (400 MHz, DMSO-d6) δ10.33 (s, 1H), 8.80 (t, 1H), 8.59 (s, 1H), 8.04 (t, 1H), 7.7 (m, 14H), 7.03 (t, 3H), 4.51 (t, 2H), 4.23 (t, 2H), 3.86 (t, 2H), 3.01 (t, 2H), and 2.07 (t, 2H). Mass spectrum identified a molecular ion at 552.35 (M+H)⁺. The purity of the compound measured by NMR was found to be >95% and by HPLC was found to be >99%. The IC50 for CREB inhibition was 0.18 μM.

The IC50 for inhibition of triple negative breast cancer breast lines MCF-7, BT-474, and Hs587T was 1.47, 5.21, and 3.69 μM, respectively. The IC50 for inhibition of leukemia cell lines KG-1, MV-411, HL-60, and NaLM6 was 3.39, 0.99, 0.65, and 0.97 μM, respectively. The IC50 for inhibition of A549, MiaPaCa2, Hep3B, and HS5 cell was 1.49, 1.70, and 1.46 μM, respectively

The compound was formulated in an aqueous solution containing 5% Tween-80, 10% DMSO. Female mice (C57BL/6) were given a single dose of the formulation at 5 mg/kg (IV), 10 mg/kg (IP) and 20 mg/kg (orally). At different time points (0.083, 0.25, 0.5, 1, 2, 4, 8, 24 h) post drug administration, blood was collected and the plasma was prepared. The samples were stored at −80° C. until analysis. The drug concentration in the plasma was analyzed by LC-MS/MS. The IV, IP, and oral bioavailability of the compound was calculated to be 100%, 100%, and 67% respectively.

### Example 7

### Synthesis of SRS-1007

The structure of the SRS-1007 is given below:

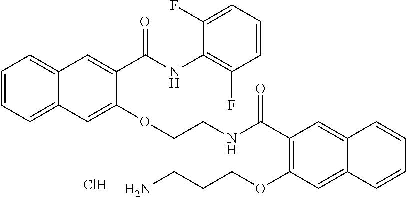 [SMILES: NCCCOc1cc2ccccc2cc1C(=O)NCCOc1cc2ccccc2cc1C(=O)Nc1c(F)cccc1F ; Cl]

The final compound was isolated as a white solid. The purity of the compound measured by NMR was found to be >95% and by HPLC was found to be >99%. The IC50 for CREB inhibition was 0.45 μM.

### Example 7

### Synthesis of SRS-1007

The structure of the SRS-1007 is given below:

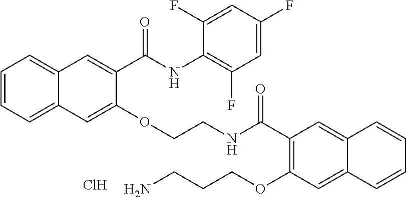 [SMILES: NCCCOc1cc2ccccc2cc1C(=O)NCCOc1cc2ccccc2cc1C(=O)Nc1c(F)cc(F)cc1F ; Cl]

The final compound was isolated as a white solid. The purity of the compound measured by NMR was found to be >95% and by HPLC was found to be >99%. The IC50 for CREB inhibition was 0.41 μM.

### Example 9

### AML Xenograft Model

For AML xenograft mouse experiments, HL-60 cells (2×10⁶) expressing firefly luciferase and GFP are injected through the tail vein into 4-6 week old NOD. Cg-Prkdc^{acid} Il2rg^{tmlWjl}SzJ (NSG) mice. Mice are then treated with 2 mg/kg SRS-1006 or 10% DMSO, injected intravenously by tail vein daily seven days after AML cell injection, until death or an endpoint is reached (moribundity) in accordance with the animal care institutional guidelines. Leukemia progression in mice at the indicated time points is monitored using an in vivo IVIS 100 bioluminescenceoptical imaging system (Xenogen Corporation). D-Luciferin (Promega) dissolved in sterile phosphate-buffered saline was injected intraperitoneally at a dose of 2.5 mg/kg, 15 minutes before measuring the luminescent signal. General anesthesia is induced with 2 isoflurane and continued during the procedure using a nose cone. Analysis is performed on Living Image In Vivo imaging software (Perkin-Elmer). Bone marrow is aspirated from bone marrow cavities, and GFP+cells is sorted using the FACSCalibur flow cytometer (BD Biosciences).

Bioluminescence imaging is performed during treatment revealed less disease burden in delayed-treated mice compared to control. CREB inhibition by SRS-1006 significantly prolonged the median survival in Kaplan-Meier analysis in the treatment groups.

### Example 10

While the invention has been particularly shown and described with reference to a preferred embodiment and various alternate embodiments, it will be understood by persons skilled in the relevant art that various changes in form and details can be made therein without departing from the spirit and scope of the invention.

We claim:

1. A compound comprising the formula: [symbol]
wherein R₁, R₂, R₃, R₄, and R₅ are independently selected to be H, Cl, F, Br, I, CN, or NO₂;
X is —NH—C(O)—, —C(O)—, or —O—;
n is 1, 2, 3, or 4; and
m is 3 or 4, and its salts, solvates, and prodrugs thereof.

2. The compound of claim 1, wherein at least one of R₁, R₂, R₃, R₄, and R₅ is Cl, F, Br, or I, and the rest are H.

3. The compound of claim 1, wherein R₁ and R₃ or R₁ and R₅ are independently selected to be Cl, F, Br, or I and the rest are H.

4. The compound of claim 3, wherein n is 2 and X is —NH—C(O)—.

5. A pharmaceutical composition comprising a compound of formula: [symbol]
wherein R₁, R₂, R₃, R₄, and R₅ are independently selected to be H, Cl, F, Br, I, CN, or NO₂;
X is —NH—C(O)—, —C(O)—, or —O—;
n is 1, 2, 3, or 4;
m is 3 or 4, and its salts, solvates, and prodrugs thereof, and a pharmaceutically acceptable carrier.

6. A method of treating or preventing a cancer in a patient in need thereof, the method comprising administering to a patient a therapeutically effective amount of a compound that inhibits cyclic-AMP (cAMP) response element binding protein (CREB).

7. The method of claim 15 wherein administering comprises oral dosage form.

8. The method of claim 15, wherein the cancer is a leukemia, breast cancer, or lung cancer.

9. The method of claim 17, wherein the patient is human.
